(19) United States
(12) Patent Application Publication
(10) Pub. No.: US 2010/0145064 A1
(43) Pub. Date: Jun. 10, 2010

(54) METHOD FOR THE PREPARATION OF ZOLMITRIPTAN

(76) Inventors: Michal Voslar, Praha (CZ); Monika Zatopkova, Ostrava (CZ); Hana Petrickova, Praha (CZ); Lucie Tisovska, Melnik (CZ)

Correspondence Address: Pearl Cohen Zedek Latzer, LLP, 1500 Broadway, 12th Floor, New York, NY, 10036, US

(21) Appl. No.: 12/527,748

(22) PCT Filed: Feb. 25, 2008
(86) PCT No.: PCT/CZ08/00022

§ 371 Date: Jan. 14, 2010

(30) Foreign Application Priority Data
Feb. 26, 2007 (CZ) PV-2007-128

(51) Int. Cl. C07D 413/12

(52) U.S. Cl. 548/229

(57) ABSTRACT

A method for the preparation of zolmitriptan or its pharmaceutically acceptable salts, comprising isolation of a crystalline salt of zolmitriptan, its optional re-purification and conversion to the substance of formula (III). Zolmitriptan hydrochloride in the crystalline state.

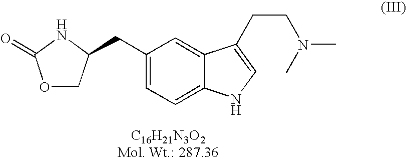

### TECHNICAL FIELD

The invention deals with a method of purification of (S)-4-[{3-[2-(dimethylamino)ethyl]-1H-indol-5-yl}methyl]-2-oxazolidinone (zolmitriptan). Zolmitriptan belongs to the group of triptans used for the treatment of migraine.

### BACKGROUND ART

Synthesis of zolmitriptan consists of three generally known reactions (WO97/06162): diazotation of (S)-4-(4-aminobenzyl)-1,3-oxazolidin-2-one of formula I with a nitrite in the environment of diluted hydrochloric acid, subsequent reduction of the resulting diazonium salt to (S)-4-(4-hydrazinobenzyl)-1,3-oxazolidin-2-one of formula IV in an acidic aqueous solution and finally its condensation with 4,4-diethoxy-N,N-dimetylbutylamine of formula II, which, in the acidic environment, according to Fischer, provides the indole skeleton with a mechanism analogous to benzidine rearrangement—in this case zolmitriptan of formula III. The whole synthesis is carried out in an aqueous environment and does not require isolation of intermediate products.

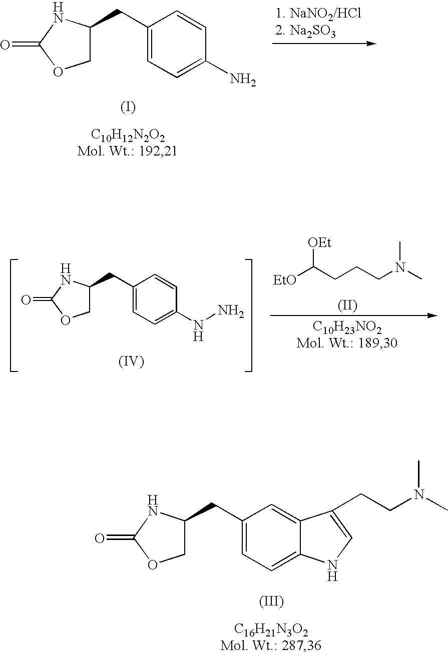

The raw product in the form of the base is usually recovered from the reaction mixture after its alkalization (NaOH/water) by repeated extraction with an organic solvent, generally ethyl acetate (WO 97/06162). After concentration of the extracts to a fraction of the initial volume crystalline raw zolmitriptan is obtained in the form of a solvate with ethyl acetate, which is purified through re-crystallization from an ethanol-ethyl acetate mixture and subsequently desolvated by stirring in aqueous acetone. The product—zolmitriptan base—is finally removed by filtration and washed with ethyl acetate.

With regard to relatively low solubility of zolmitriptan in ethyl acetate the above mentioned extraction method of isolation requires considerable volumes of the extraction agent (ca. 25-fold of the final product) and their subsequent evaporation. To say nothing of the energy and time demands of such a method, during concentrating in a larger scale the product is also subject to considerable heat load which is, with regard to the increased temperature and character of the solvent, even multiplied by the mutual reactivity of the product (basic amine) with the solvent (ester) and also by hydrolysis of the product and solvent caused by the alkaline aqueous environment during the extractions!

### DISCLOSURE OF INVENTION

The invention consists in a new method for the preparation of (S)-4-[{3-[2-(dimethylamino)ethyl]-1H-indol-5-yl}methyl]-2-oxazolidinone (zolmitriptan), which comprises isolation of a crystalline salt, especially of zolmitriptan hydrochloride. Another aspect of the invention includes the crystalline hydrochloride of (S)-4-[{3-[2-(dimethylamino)ethyl]-1H-indol-5-yl}methyl]-2-oxazolidinone (zolmitriptan), which can be used as a useful intermediate of the synthesis, as well as an effective component of a pharmaceutically useful composition.

### DESCRIPTION OF INVENTION

It has been demonstrated experimentally that raw zolmitriptan hydrochloride, prepared by condensation of (S)-4-(4-hydrazinobenzyl)-1,3-oxazolidin-2-one of formula IV with 4,4-diethoxy-N,N-dimetylbutyl-amine of formula II in an acidic aqueous environment, can be preferably salted out at a reduced temperature by a considerable increase of the ion strength of the mixture, namely by adding a salt from the group of alkali metal chlorides, preferably NaCl.

Crystalline zolmitriptan hydrochloride is a stable salt, which can be transformed to the free base that is commonly used in the pharmaceutical industry or used directly for the preparation of the medicament, or transformed to another pharmaceutically acceptable salt.

Suitable for the purposes of efficient purification of the product is the crystalline form, characterized by the X-ray diffraction pattern (λCuKα=1.5418 Å) in the range of 4-40° 2θ with the increment of 0.008 and the typical 2 theta signals: 7.36; 17.91; 18.59; 19.17; 19.40; 23.05.

The crystalline form the reflections of which are summarized in the table below appears to be especially advantageous:

TABLE 1
Zolmitriptan•HCl

| 2 theta | d | Irel |
|---|---|---|
| 7.12 | 12.40 | 14.86 |
| 7.36 | 12.00 | 23.61 |
| 12.96 | 6.83 | 12.05 |
| 15.78 | 5.61 | 28.88 |
| 16.01 | 5.53 | 33.55 |
| 17.91 | 4.95 | 83.08 |
| 18.27 | 4.85 | 52.77 |
| 18.59 | 4.77 | 96.48 |
| 18.81 | 4.71 | 39.52 |
| 19.17 | 4.63 | 100.00 |
| 19.40 | 4.57 | 58.44 |
| 21.58 | 4.12 | 27.54 |
| 21.99 | 4.04 | 51.71 |
| 22.43 | 3.96 | 33.89 |
| 23.05 | 3.86 | 64.52 |
| 23.33 | 3.81 | 20.69 |
| 25.03 | 3.56 | 8.74 |
| 25.18 | 3.53 | 12.89 |
| 25.37 | 3.51 | 11.50 |
| 25.89 | 3.44 | 9.10 |
| 26.09 | 3.41 | 17.94 |
| 26.46 | 3.37 | 10.71 |
| 26.63 | 3.34 | 10.51 |
| 28.16 | 3.17 | 7.67 |
| 28.71 | 3.11 | 10.13 |
| 29.32 | 3.04 | 8.67 |
| 30.16 | 2.96 | 11.01 |
| 30.24 | 2.95 | 9.16 |
| 31.69 | 2.82 | 9.29 |
| 36.65 | 2.45 | 11.37 |

The crystalline form of zolmitriptan hydrochloride prepared in the above-described preferable method is further characterized by a melting temperature in the interval of 148 to 154° C. and also by an endotherm with the peak at 142±3° C. and the onset at 127±3° C., measured in an N₂ stream, in a DSC record at the heating rate of 10° C./min.

Zolmitriptan in the free base form, which is insoluble in water, is released from the salt by gradual addition of an alkali metal carbonate or hydroxide, e.g. K₂CO₃ or NaOH, etc., to an aqueous solution of the hydrochloride or its solution in an alcohol-water mixture. The base is removed by filtration and washed with aqueous alcohol and water, or it is stirred up in purified water. After drying at temperatures of about 45° C. the obtained zolmitriptan in the base form can be optionally re-purified by re-crystallization from an ethanol-water mixture. The resulting white or almost white product (HPLC ˜100.0%) corresponds, according to the X-ray analysis, to form A, the same one as can be prepared with the method in accordance with patent application no. WO 97/06162.

Advantages of the above mentioned newly developed isolation and purification process include the speed, low price of input materials, a high yield and quality of the obtained product and, above all, minimum impacts on the environment since except a small volume of ethanol no organic solvents are used in the whole process. At the same time the process has managed to remove the above mentioned problems that accompany the common isolation method based on extractions of the product with ethyl acetate.

### BRIEF DESCRIPTION OF DRAWINGS

FIG. 1 shows an X-ray diffraction pattern of the HCl salt of zolmitriptan.

FIG. 2 shows a DSC record of the HCl salt of zolmitriptan.

### EXAMPLES

### Example 1

### Fischer Indole Reaction

The diluted reaction mixture containing (S)-4-(4-hydrazinobenzyl)-1,3-oxazolidin-2-one prepared by diazotation 1.75 kg of the amine (I) and reduction in accordance with the above mentioned scheme is heated up to ca. 90° C. 1.75 kg of 4,4-diethoxy-N,N-dimethylbutylamine of formula II is weighed. The weighed acetal (II) is added to the reaction mixture, which is then brought to moderate reflux at ca. 98° C. and left to react being stirred under a reflux condenser for 2.5 hours. After 2.5 hours from the start of the reflux the heating of the mixture is switched off and the reaction mixture is cooled to the laboratory temperature.

### Example 2

### Isolation of Raw ZOLMITRIPTAN Hydrochloride

The reaction mixture is cooled down to ca. 10° C. and about 9 kg of NaCl is added during stirring. The mixture is stirred at this temperature for another ca. 45 min. The salted out zolmitriptan hydrochloride is then removed by filtration and washed with ca. 1.7 litres of saturated NaCl solution.

### Example 3

### Transforming the ZOLMITRIPTAN Hydrochloride to the Base

The zolmitriptan hydrochloride salted out from the reaction mixture in accordance with Example 4 is dissolved in ca. 12 litres of water under stirring and about 2.5 litres of ethanol are added. 1.0 kg of potassium carbonate is added in several doses to the solution under stirring at the temperature of ca. 25° C. 30 mins after the last addition the suspension of the product is cooled to 10-15° C. while being stirred and the product is removed by filtration after approximately 1 hour of stirring. The filtration cake is repeatedly washed with ca. 2 litres of purified water (or the filtered base is re-suspended and stirred up in purified water) and left to dry under normal pressure at the temperature of about 45° C. to the constant weight.

The product may be optionally re-purified by re-crystallization from an ethanol-water mixture.

### Example 4

### Purification of ZOLMITRIPTAN Hydrochloride

Raw zolmitriptan hydrochloride is re-purified by conversion to the base in aqueous alcohol in accordance with Example 3. Under stirring the base is then dissolved in ca. 8 litres of 2% HCl and solid zolmitriptan hydrochloride is salted out again from this aqueous solution by the addition of about 2 kg of NaCl at the temperature of ca. 10° C. Approximately after 30 minutes of stirring the product is removed by filtration and washed with ice-cold water, or stirred up in ice-cold water. Pure zolmitriptan hydrochloride is dried in vacuo at the laboratory temperature for about 8 hours and then to the constant weight at ca. 50° C.

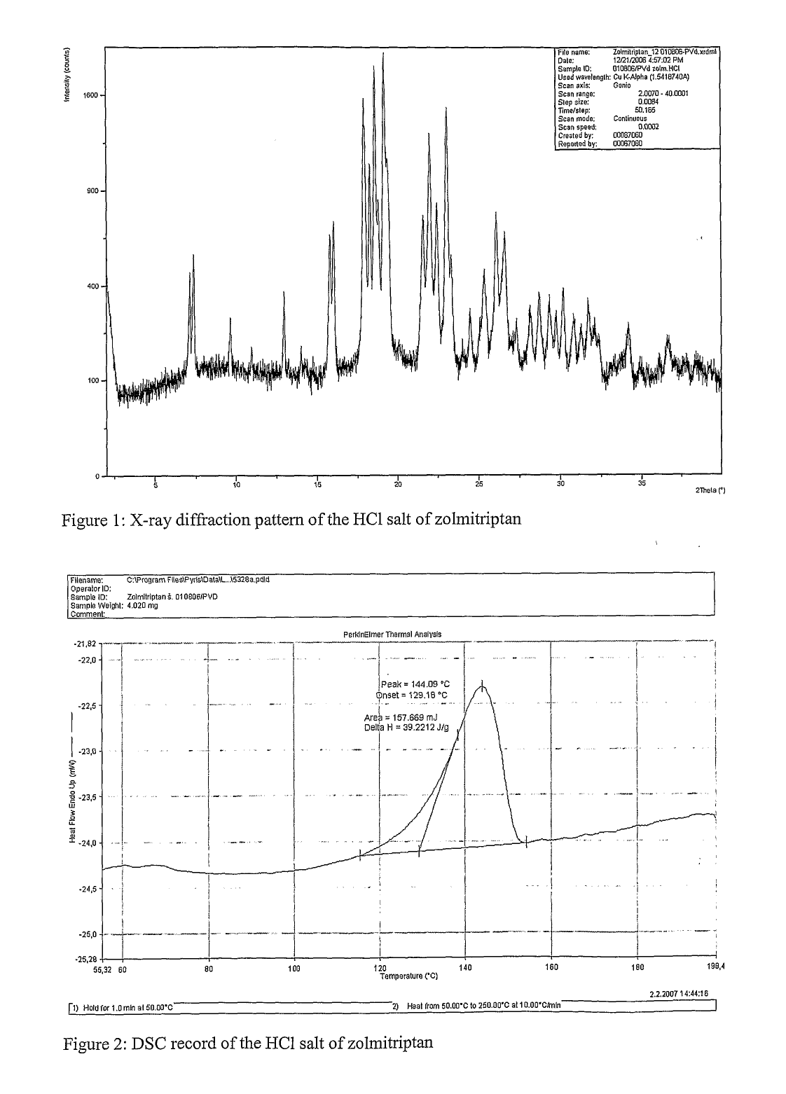

The obtained zolmitriptan hydrochloride salt was characterized by the X-ray powder diffraction method (FIG. 1) and the DSC method (FIG. 2).

1. A method for the preparation of (S)-4-[{3-[2-(dimethylamino)ethyl]-1H-indol-5-yl}methyl]-2-oxazolidinone (zolmitriptan) of formula III 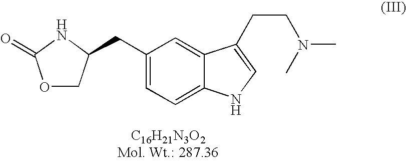
or its pharmaceutically acceptable salts, prepared by isolation of a crystalline salt of zolmitriptan re-purification, and conversion converted into the substance of formula III.

2. The method in accordance with claim 1, wherein the conversion of the salt to the substance of formula III is carried out in an aqueous solution or in an alcohol-water mixture by alkali metal carbonates or hydroxides.

3. The method in accordance with claim 1, wherein the crystalline zolmitriptan hydrochloride is isolated by salting out from an aqueous solution by the effect of an alkali metal chloride.

4. The method of claim 1 comprising the steps of:
a) Preparing zolmitriptan hydrochloride by reacting (S)-4-(4-hydrazinobenzyl)-1,3-oxazolidin-2-one hydrochloride with 4,4-diethoxy-N,N-dimethylbutylamine by the Fischer indole reaction;
b) Salting out the zolmitriptan hydrochloride from the reaction mixture after the Fischer indole synthesis, at a reduced temperature, by adding a salt which is an alkali metal chloride;
c) Re-purifying the raw zolmitriptan hydrochloride by conversion into a base by adding an alkali metal carbonate or hydroxide, in aqueous alcohol, and recovering an aqueous solution of the hydrochloride by dissolution of the filtered base in a diluted acid;
d) Isolating the pure zolmitriptan hydrochloride in the crystalline form by salting out from the acidic aqueous solution;
e) Desalting of zolmitriptan hydrochloride by stirring up in ice-cold water; and
f) Converting the hydrochloride to the substance of formula III by an alkali metal carbonate or hydroxide.

5. The method in accordance with claim 1, further comprising converting the zolmitriptan hydrochloride to a pharmaceutically acceptable salt.

6. A crystalline zolmitriptan hydrochloride.

7. The crystalline zolmitriptan hydrochloride in accordance with claim 6, having a melting temperature in the interval of 148-154° C.

8. The crystalline zolmitriptan hydrochloride in accordance with claim 6, characterized by a DSC record displaying one endotherm with the peak at 142±3° C. and onset at 127±3° C. in the measured interval of 50-200° C. at the heating rate of 10° C./min, measured in an N₂ stream.

9. The crystalline zolmitriptan hydrochloride in accordance with claim 6, characterized by the X-ray difractogram (λCuKα=1.5418 Å) in the range of 4-40° with the increment of 0.008 with the typical 2 theta signals: 7.36; 17.91; 18.59; 19.17; 19.40; 23.05.
